(19) United States
(12) Patent Application Publication
(10) Pub. No.: US 2018/0282818 A1
(43) Pub. Date: Oct. 4, 2018

(54) Therapeutic Cancer Treatments Based on TP53 Gene Mutations

(71) Applicant: The Wistar Institute of Anatomy and Biology, Philadelphia, PA (US)

(72) Inventor: Maureen E. Murphy, Glenside, PA (US)

(21) Appl. No.: 15/764,969

(22) PCT Filed: Oct. 7, 2016
(86) PCT No.: PCT/US16/56077

§ 371 Date: Mar. 30, 2018

Related U.S. Application Data
(60) Provisional application No. 62/239,250, filed on Oct. 8, 2015.

(51) Int. Cl. C12Q 1/6886; A61K 31/53; A61K 31/519; A61K 31/501; A61K 31/5377; A61K 33/24; A61K 31/704; A61K 31/4045; A61K 31/506

(52) U.S. Cl. CPC C12Q 1/6886; A61K 31/53; A61K 31/519; A61K 31/501; C12Q 2600/156; A61K 33/24; A61K 31/704; A61K 31/4045; A61K 31/506; A61K 31/5377

(57) ABSTRACT

Therapeutic treatments for cancer based on mutations in the TP53 gene are disclosed, including pharmaceutical corn-positions and methods of using pharmaceutical compositions for treating a disease, in particular a cancer. Diagnostic methods for determining mutations in the TP53 gene are also disclosed.

### CROSS-REFERENCE TO RELATED APPLICATIONS

The application claims the benefit of priority to U.S. Provisional Patent Application No. 62/239,250, filed Oct. 8, 2015, the entirety of which is incorporated herein by reference.

### GOVERNMENT SUPPORT

This invention was made with Government support under NIH Grant No. R01 CA102184 and NCI Grant No. P30 CA010815, awarded by the National Institutes of Health. The government has certain rights in the invention.

### FIELD OF THE INVENTION

Therapeutic treatments for cancer based on mutations in the TP53 gene are disclosed. In addition, diagnostic methods for determining mutations in the TP53 gene are disclosed.

### BACKGROUND OF THE INVENTION

The p53 tumor suppressor (TP53) gene is frequently mutated in human cancers. For example, germline mutations in the TP53 gene are related to Li Fraumeni syndrome, which causes tumors of the brain, breast, bone and adrenal cortex. Malkin, et al., N. Engl. J. Med. 1992, 326, 1309-1315. Somatic mutations in the TP53 gene account for sixty percent of sporadic human tumors. Hollstein, et al., Science 1991, 253, 49-53. Most p53 mutations in human tumors are classified as DNA binding domain missense mutations and inhibit protein binding to p53 response elements in the promoters of p53 target genes, resulting in transactivation of gene expression. Vogelstein, et al., Nature 2000, 408, 307-310.

p53 can make use of at least three different subsets of target genes to suppress tumor development, through transcriptional, apoptotic and tumor suppressor mechanisms. Brady, et al., Cell 2011, 145, 571-583; Li, et al., Cell, 2012, 149, 1269-1283; Schmitt, et al., Cancer Cell 2002, 1, 289-298. Post-translational modification of p53 has a significant impact on its ability to perform its functions. Kruse and Gu, Cell 2008, 133, 930-930; Vousden and Prives, Cell 2009, 137, 413-431. For example, serine 46 phosphorylation is required for efficient p53-mediated apoptosis in several cell lines. Bulavin, et al., EMBO J. 1999, 18, 6845-6854; D'Orazi, et al., Nat. Cell. Biol. 2002, 4, 11-19; Oda, et al., Cell 2000, 102, 849-862.

A naturally-occurring polymorphism in TP53 exists in African and Hispanic populations (r51800371, G/A) that converts the proline residue proximal to Serine 46 in human p53 to a serine (P47S). This replacement eliminates the proline required for phosphorylation of Serine 46 by the proline-directed kinases p38MAPK, HIPK2 and DYRK. The Serine 47 (S47) variant is markedly impaired with respect to phosphorylation on serine 46, and has significantly impaired apoptotic ability in multiple human cell lines engineered to contain inducible human forms of p53. Li, et al., J. Biol. Chem. 2005, 280, 24245-24251. However, the impact of the S47 variant on cancer risk in humans, and the efficacy of various treatment options for patients with this variant, is unknown.

Amongst large racial and ethnic groups in the United States, African Americans exhibit the highest mortality rate and shortest survival for many cancers. The genetic basis for this disparity in mortality rate and survival for this ethnic group has thus far been elusive. Genetic admixture mapping has identified deleterious loci on 8q24 in prostate cancer risk in African Americans, but the genes responsible have not been identified. Bock, et al., Hum. Genet. 2009, 126, 637-642; Xu, et al., Cancer Epidemiol. Biomarkers Prev. 2009, 18, 2145-2149. The P47S polymorphism was previously identified as existing in African Americans. Felley-Bosco, et al., Am. J. Hum. Genet. 1993, 53, 752-759.

The PI3K signal transduction pathway is known to be highly mutated in human cancers, and signaling through this pathway is a key factor in hematologic malignancies and inflammatory diseases. The role of PI3K in cancer has been discussed, for example, in Engleman, Nat. Rev. Cancer 2009, 9, 550-562. Numerous isoforms of PI3K are known, including the PI3K-α, PI3K-β, PI3K-γ, and PI3K-δ isoforms. Vanhaesebroeck, et al., Nature Rev. Mol. Cell Biol. 2010, 11, 329-341. Downstream mediators of the PI3K signal transduction pathway include AKT and mammalian target of rapamycin (mTOR). mTOR is a serine-threonine kinase related to the lipid kinases of the PI3K family. mTOR has been implicated in a wide range of biological processes including cell growth, proliferation, metabolism, and survival, and disregulation of the mTOR pathway has been reported in various types of cancer. Guertin and Sabatini, Cancer Cell, 2007, 12, 9-22. mTOR forms two distinct multiprotein complexes, mTORC1 and mTORC2. Therapies that target the PI3K and mTOR pathways are urgently needed for the treatment of cancer, inflammatory diseases, autoimmune diseases, and other diseases.

The invention provides the unexpected finding that the P47S polymorphism in TP53 has a major impact on the ability of this protein to induce apoptosis, transactivate target genes, and suppress tumor development, and further provides that certain cancer patient subpopulations may be more efficaciously treated than others based on the particular cancer and the presence or absence of the P47S polymorphism. The invention further provides the unexpected finding that active pharmaceutical ingredients including kinase inhibitors, antineoplastic agents, and chemotherapeutic agents are synergistically effective in the treatment of any of several subtypes of cancers, such as solid tumor cancers wherein a biological sample exhibits the P47S polymorphism.

### SUMMARY OF THE INVENTION

In an embodiment, the invention includes a method of treating a cancer in a human subject with an inherited non-synonymous single nucleotide polymorphism (SNP) at codon 47 in a TP53 gene comprising administering a therapeutically effective dose of an active pharmaceutical ingredient to the human subject, where the subject is either homozygous or heterozygous for the SNP.

In an embodiment, the invention includes a method of treating a cancer in a human subject with an inherited non-synonymous single nucleotide polymorphism at codon 47 in a TP53 gene comprising administering a therapeutically effective dose of an active pharmaceutical ingredient to the human subject, where the subject is either homozygous or heterozygous for the SNP, wherein the active pharmaceutical ingredient is selected from the group consisting of PI3K inhibitors (including PI3K-γ/δ inhibitors and PI3K-α, -β, -γ, and -δ inhibitors), mTOR inhibitors, platinum drugs, and combinations thereof.

In an embodiment, the invention includes a method of treating a cancer in a human subject with an inherited non-synonymous single nucleotide polymorphism at codon 47 in a TP53 gene comprising administering a therapeutically effective dose of an active pharmaceutical ingredient to the human subject, where the subject is either homozygous or heterozygous for the SNP, wherein the cancer is a cancer that does not mutate the TP53 gene.

In an embodiment, the invention includes a method of treating a cancer in a human subject with an inherited non-synonymous single nucleotide polymorphism at codon 47 in a TP53 gene comprising administering a therapeutically effective dose of an active pharmaceutical ingredient to the human subject, where the subject is either homozygous or heterozygous for the SNP, wherein the cancer is selected from the group consisting of cancers that rarely mutate p53, wherein the group of cancers that rarely mutate p53 consists of melanoma, medulloblastoma, Wilms tumor, neuroblastoma, colorectal cancer, breast cancer, prostate cancer, and liver cancer.

In an embodiment, the invention includes a method of treating a cancer in a human subject with an inherited non-synonymous single nucleotide polymorphism at codon 47 in a TP53 gene comprising administering a therapeutically effective dose of an active pharmaceutical ingredient to the human subject, where the subject is either homozygous or heterozygous for the SNP, wherein the human subject is of Hispanic or African-American origin.

In an embodiment, the invention includes a method of treating a cancer in a human subject with an inherited non-synonymous single nucleotide polymorphism at codon 47 in a TP53 gene comprising administering a therapeutically effective dose of an active pharmaceutical ingredient to the human subject, where the subject is either homozygous or heterozygous for the SNP, wherein the active pharmaceutical ingredient is a mTOR inhibitor, and wherein the mTOR inhibitor is selected from the group consisting of trans-4-[4-amino-5-(7-methoxy-1H-indol-2-yl)imidazo[5,1-f][1,2,4]triazin-7-yl]cyclohexanecarboxylic acid (OSI-027), sapanisertib, omipalisib, dactolisib, everolimus, temsirolimus, sirolimus, and pharmaceutically acceptable salts, solvates, hydrates, cocrystals, or prodrugs thereof.

In an embodiment, the invention includes a method of treating a cancer in a human subject with an inherited non-synonymous single nucleotide polymorphism at codon 47 in a TP53 gene comprising administering a therapeutically effective dose of an active pharmaceutical ingredient to the human subject, where the subject is either homozygous or heterozygous for the SNP, wherein the active pharmaceutical ingredient is a PI3K inhibitor, and wherein the PI3K inhibitor is selected from the group consisting of omipalisib, dactolisib, pictilisib, buparlisib, duvelisib, copanlisib, idelalisib, and pharmaceutically acceptable salts, solvates, hydrates, cocrystals, or prodrugs thereof.

In an embodiment, the invention includes a method of treating a cancer in a human subject with an inherited non-synonymous single nucleotide polymorphism at codon 47 in a TP53 gene comprising administering a therapeutically effective dose of an active pharmaceutical ingredient to the human subject, where the subject is either homozygous or heterozygous for the SNP, wherein the active pharmaceutical ingredient is a platinum drug, and wherein the platinum drug is selected from the group consisting of cisplatin, carboplatin, oxaliplatin, satraplatin, picoplatin, nedaplatin, triplatin tetranitrate, lipoplatin (liposomal cisplatin), and pharmaceutically acceptable salts, solvates, hydrates, cocrystals, or prodrugs thereof.

In an embodiment, the invention includes a method performed on a biological sample from a human subject, comprising detecting a non-synonymous single nucleotide polymorphism at codon 47 in a TP53 gene in the biological sample.

In an embodiment, the invention includes a method performed on a biological sample from a human subject, comprising detecting a non-synonymous single nucleotide polymorphism at codon 47 in a TP53 gene in the biological sample, wherein the detecting further comprises detecting the presence of a nucleic acid sequence. In some embodiments, detecting may include using a detector sequence in a diagnostic PCR test that may be complimentary to the nucleic acid sequence. In some embodiments, the detector sequence may be used to determine the absence or presence of rs1800371.

In an embodiment, the invention includes a method performed on a biological sample from a human subject, comprising detecting a non-synonymous single nucleotide polymorphism at codon 47 in a TP53 gene in the biological sample, wherein the detecting further comprises amplifying the nucleic acid sequence.

In an embodiment, the invention includes a method performed on a biological sample from a human subject, comprising detecting a non-synonymous single nucleotide polymorphism at codon 47 in a TP53 gene in the biological sample, wherein the detecting comprises quantitatively analyzing the non-synonymous single nucleotide polymorphism.

In an embodiment, the invention includes a method performed on a biological sample from a human subject, comprising detecting a non-synonymous single nucleotide polymorphism at codon 47 in a TP53 gene in the biological sample, wherein the detecting comprises quantitatively analyzing the non-synonymous single nucleotide polymorphism, wherein the quantitatively analyzing comprises quantitatively analyzing by a quantitative reverse-transcription polymerase chain reaction.

In an embodiment, the invention includes a method performed on a biological sample from a human subject, comprising detecting a non-synonymous single nucleotide polymorphism at codon 47 in a TP53 gene in the biological sample, wherein the biological sample is a blood sample.

In an embodiment, the invention includes a composition comprising therapeutically effective amounts of an active pharmaceutical ingredient selected from the group consisting of a PI3K inhibitor, a mTOR inhibitor, a platinum drug, and a combination thereof, for use in the treatment of a cancer in a human with an inherited non-synonymous single nucleotide polymorphism at codon 47 in a TP53 gene.

In an embodiment, the invention includes a composition comprising therapeutically effective amounts of an active pharmaceutical ingredient selected from the group consisting of a PI3K inhibitor, a mTOR inhibitor, a platinum drug, and a combination thereof, for use in the treatment of a cancer in a human with an inherited non-synonymous single nucleotide polymorphism at codon 47 in a TP53 gene, wherein the cancer is a cancer that does not mutate the TP53 gene.

In an embodiment, the invention includes a composition comprising therapeutically effective amounts of an active pharmaceutical ingredient selected from the group consisting of a PI3K inhibitor, a mTOR inhibitor, a platinum drug, and a combination thereof, for use in the treatment of a cancer in a human with an inherited non-synonymous single nucleotide polymorphism at codon 47 in a TP53 gene, wherein the cancer is selected from the group consisting of cancers that rarely mutate p53, wherein the group of cancers that rarely mutate p53 consists of melanoma, medulloblastoma, Wilms tumor, neuroblastoma, colorectal cancer, breast cancer, prostate cancer, and liver cancer.

In an embodiment, the invention includes a composition comprising therapeutically effective amounts of an active pharmaceutical ingredient selected from the group consisting of a PI3K inhibitor, a mTOR inhibitor, a platinum drug, and a combination thereof, for use in the treatment of a cancer in a human with an inherited non-synonymous single nucleotide polymorphism at codon 47 in a TP53 gene, wherein the human subject is of Hispanic or African-American origin.

In an embodiment, the invention includes a composition comprising therapeutically effective amounts of an active pharmaceutical ingredient selected from the group consisting of a PI3K inhibitor, a mTOR inhibitor, a platinum drug, and a combination thereof, for use in the treatment of a cancer in a human with an inherited non-synonymous single nucleotide polymorphism at codon 47 in a TP53 gene, wherein the active pharmaceutical ingredient is a mTOR inhibitor, and wherein the mTOR inhibitor is selected from the group consisting of trans-4-[4-amino-5-(7-methoxy-1H-indol-2-yl)imidazo[5,1-f][1,2,4]triazin-7-yl]cyclohexanecarboxylic acid (OSI-027), sapanisertib, omipalisib, dactolisib, everolimus, temsirolimus, sirolimus, and pharmaceutically acceptable salts, solvates, hydrates, cocrystals, or prodrugs thereof.

In an embodiment, the invention includes a composition comprising therapeutically effective amounts of an active pharmaceutical ingredient selected from the group consisting of a PI3K inhibitor, a mTOR inhibitor, a platinum drug, and a combination thereof, for use in the treatment of a cancer in a human with an inherited non-synonymous single nucleotide polymorphism at codon 47 in a TP53 gene, wherein the active pharmaceutical ingredient is a PI3K inhibitor, and wherein the PI3K inhibitor is selected from the group consisting of omipalisib, dactolisib, pictilisib, buparlisib, duvelisib, copanlisib, idelalisib, and pharmaceutically acceptable salts, solvates, hydrates, cocrystals, or prodrugs thereof.

In an embodiment, the invention includes a composition comprising therapeutically effective amounts of an active pharmaceutical ingredient selected from the group consisting of a PI3K inhibitor, a mTOR inhibitor, a platinum drug, and a combination thereof, for use in the treatment of a cancer in a human with an inherited non-synonymous single nucleotide polymorphism at codon 47 in a TP53 gene, wherein the active pharmaceutical ingredient is a platinum drug, and wherein the platinum drug is selected from the group consisting of cisplatin, carboplatin, oxaliplatin, satraplatin, picoplatin, nedaplatin, triplatin tetranitrate, lipoplatin (liposomal cisplatin), and pharmaceutically acceptable salts, solvates, hydrates, cocrystals, or prodrugs thereof.

### BRIEF DESCRIPTION OF THE DRAWINGS

The foregoing summary, as well as the following detailed description of the invention, will be better understood when read in conjunction with the appended drawings.

FIG. 1 illustrates hematoxylin and eosin staining of tumors from the S47 mouse. Left: Normal liver and liver hepatocellular carcinoma (HCC, dotted outline). Right: infiltrating B cell lymphoma in the kidney (arrows). Scale bar represents 100 μm.

FIG. 2 illustrates hematoxylin and eosin staining of tumors from the S47 mouse. Upper left: HCC. Upper right: HCC metastasized to the lung (dotted outline). Middle left: pancreatic adenocarcinoma (asterisks). Middle right: pancreatic adenocarcinoma metastasized to a lymph node. Lower left: intestinal adenoma. Lower right: stomach adenoma. Scale bar represents 100 μm.

FIG. 3 illustrates Kaplan-Meier analysis of survival between WT and S47 mice (n=20 each). The asterisk represents a single wild type (WT) mouse that died from a non-cancerous cause.

FIG. 4 illustrates metabolism genes with impaired induction in S47 cells (human lymphoblastoid cells). Cells with WT p53 or homozygous S47 were treated with 10 μM cisplatin for 0, 8 and 24 hours, and the genes listed were found to have impaired transactivation in S47.

FIG. 5 illustrates impaired oxidative phosphorylation in S47 cells, compared to cells with WT p53. Analysis of oxygen consumption rate (upper left) in WT and S47 cells indicates impaired OCR in S47 cells (top panels). Also plotted are the maximum respiratory capacity and extra-cellular acidification rate; the ECR is indicative of lactic acid production from aerobic glycolysis. Data are the averages of three independent experiments.

FIG. 6 illustrates that S47 tumor cells are more sensitive to metabolic poisons like the mTOR inhibitor OSI-027. S47 and WT cells expressing E1A/Ras were analyzed.

FIG. 7 illustrates that S47 tumor cells are less sensitive to camptothecin and etoposide (topoisomerase inhibitors).

### BRIEF DESCRIPTION OF THE SEQUENCE LISTING

SEQ ID NO:1 is a nucleotide sequence for use in a diagnostic PCR test for determining the absence or presence of rs1800371.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

“Prodrug” is intended to describe a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers the advantages of solubility, tissue compatibility or delayed release in a mammalian organism (see, e.g., Bundgaard, H., Design of Prodrugs (1985) (Elsevier, Amsterdam). The term “prodrug” is also intended to include any covalently bonded carriers, which release the active compound in vivo when administered to a subject. Prodrugs of an active compound, as described herein, may be prepared by modifying functional groups present in the active compound in such a way that the modifications are cleaved, either in routine manipulation or in vivo, to yield the active parent compound. Prodrugs include, for example, compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetates, formates and benzoate derivatives of an alcohol, various ester derivatives of a carboxylic acid, or acetamide, formamide and benzamide derivatives of an amine functional group in the active compound.

As used herein, the term “warhead” or “warhead group” refers to a functional group present on a compound of the invention wherein that functional group is capable of covalently binding to an amino acid residue present in the binding pocket of the target protein (such as cysteine, lysine, histidine, or other residues capable of being covalently modified), thereby irreversibly inhibiting the protein.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this invention.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (e.g., (C₁₋₁₀ )alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocycyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂ N(R^{a}) (NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group -O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., -O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)—C(O)—, (aryl)—C(O)—, (heteroaryl)—C(O)—, (heteroalkyl)—C(O)— and (heterocycloalkyl)—C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or O. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In preferred embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, N.Y. (1981); E. L. Eliel, Stereochemistry of Carbon Compounds, McGraw-Hill, N.Y. (1962); and E. L. Eliel and S. H. Wilen, Stereochemistry of Organic Compounds, Wiley-Interscience, N.Y. (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, N.Y. (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the invention also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### Methods of Treating Cancers and Other Diseases

The compositions and methods described herein can be used in a method for treating diseases. In a preferred embodiment, they are for use in treating hyperproliferative disorders. They may also be used in treating other disorders as described herein and in the following paragraphs.

In some embodiments, the hyperproliferative disorder is cancer. In selected embodiments, the cancer is selected from the group consisting of non-Hodgkin's lymphomas (such as diffuse large B-cell lymphoma), acute myeloid leukemia, thymus, brain, lung, squamous cell, skin, eye, retinoblastoma, intraocular melanoma, oral cavity and oropharyngeal, bladder, gastric, stomach, pancreatic, bladder, breast, cervical, head, neck, renal, kidney, liver, ovarian, prostate, colorectal, bone (e.g., metastatic bone), esophageal, testicular, gynecological, thyroid, CNS, PNS, AIDS-related (e.g. lymphoma and Kaposi's sarcoma), viral-induced cancers such as cervical carcinoma (human papillomavirus), B-cell lymphoproliferative disease and nasopharyngeal carcinoma (Epstein-Barr virus), Kaposi's sarcoma and primary effusion lymphomas (Kaposi's sarcoma herpesvirus), hepatocellular carcinoma (hepatitis B and hepatitis C viruses), and T-cell leukemias (Human T-cell leukemia virus-1), B cell acute lymphoblastic leukemia, Burkitt's leukemia, juvenile myelomonocytic leukemia, hairy cell leukemia, Hodgkin's disease, multiple myeloma, mast cell leukemia, and mastocytosis. In selected embodiments, the method relates to the treatment of a non-cancerous hyperproliferative disorder such as benign hyperplasia of the skin (e.g., psoriasis), restenosis, or prostate conditions (e.g., benign prostatic hypertrophy (BPH)).

Efficacy of the compounds and combinations of compounds described herein in treating, preventing and/or managing the indicated diseases or disorders can be tested using various models known in the art, which provide guidance for treatment of human disease. For example, models for determining efficacy of treatments for pancreatic cancer are described in Herreros-Villanueva, et al., World J. Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for ovarian cancer are described, e.g., in Mullany, et al., Endocrinology 2012, 153, 1585-92; and Fong, et al., J. Ovarian Res. 2009, 2, 12. Models for determining efficacy of treatments for melanoma are described, e.g., in Damsky, et al., Pigment Cell & Melanoma Res. 2010, 23, 853-859. Models for determining efficacy of treatments for lung cancer are described, e.g., in Meuwissen, et al., Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1, 32. Models for determining efficacy in B cell lymphomas, such as diffuse large B cell lymphoma (DLBCL), include the PiBCL1 murine model with BALB/c (haplotype H-2d) mice. Illidge, et al., Cancer Biother. & Radiopharm. 2000, 15, 571-80. Efficacy of treatments for Non-Hodgkin's lymphoma (NHL) may be assessed using the 38C13 murine model with C3H/HeN (haplotype 2-Hk) mice or alternatively the 38C13 Her2/neu model. Timmerman, et al., Blood 2001, 97, 1370-77; Penichet, et al., Cancer Immunolog. Immunother. 2000, 49, 649-662. Efficacy of treatments for chronic lymphocytic leukemia (CLL) may be assessed using the BCL1 model using BALB/c (haplotype H-2d) mice. Dutt, et al., Blood 2011, 117, 3230-29. Models for other cancers are known to those of ordinary skill in the art.

mTOR/PI3K Inhibitors

In an embodiment, the invention includes a method of treating a cancer in a human subject with an inherited non-synonymous single nucleotide polymorphism at codon 47 in a TP53 gene comprising administering a therapeutically effective dose of an active pharmaceutical ingredient to the human subject, wherein the active pharmaceutical ingredient is selected from the group consisting of a PI3K inhibitor, a mTOR inhibitor, and a combination thereof. The active pharmaceutical ingredient may be any PI3K inhibitor or mTOR inhibitor known in the art. Suitable mTOR inhibitors are described, for example, in Verheij en, et al., Ann. Rep. Med. Chem. 2008, 43, 189-202. In particular, it is one of the PI3K inhibitors and mTOR inhibitors described in more detail in the following paragraphs.

In an embodiment, the mTOR inhibitor is trans-4-[4-amino-5-(7-methoxy-1H-indol-2-yl)imidazo[5,1-f][1,2,4]triazin-7-yl]cyclohexanecarboxylic acid, also known as OSI-02 (Formula (1)):

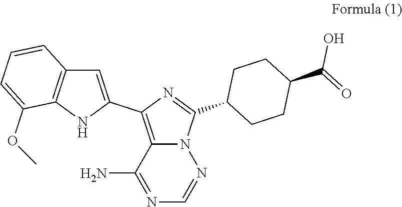 [SMILES: COc1cccc2cc(-c3nc([C@H]4CC[C@H](C(=O)O)CC4)n4ncnc(N)c34)[nH]c12]

or a pharmaceutically acceptable salt, solvate, hydrate, cocrystal, or prodrug thereof. Formula (1) is a commercially-available mTOR kinase inhibitor. Formula (1) may be synthetically prepared using methods disclosed in U.S. Patent Application Publication No. US 2007/112005, the disclosure of which is incorporated by reference herein. The properties of Formula (1) are described in Bhagwat, et al., Mol. Cancer. Ther. 2011, 10, 1394-1406.

In an embodiment, the mTOR inhibitor is sapanisertib, which has the chemical name 5-(4-amino-1-isopropyl-1H-pyrazolo[3 ,4-d]pyrimidin-3 -yl)benzo[d]oxazol-2-amine, and which is also known as INK128 (Formula (2)):

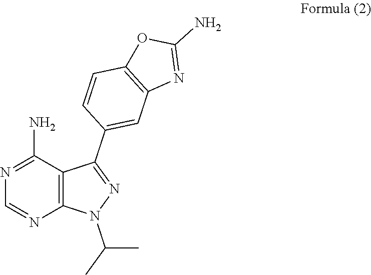 [SMILES: CC(C)n1nc(-c2ccc3oc(N)nc3c2)c2c(N)ncnc21]

or a pharmaceutically acceptable salt, solvate, hydrate, cocrystal, or prodrug thereof. Sapanisertib is a commercially-available mTOR kinase inhibitor.

In an embodiment, the mTOR inhibitor is everolimus, also known as RAD-001 (Formula (3)):

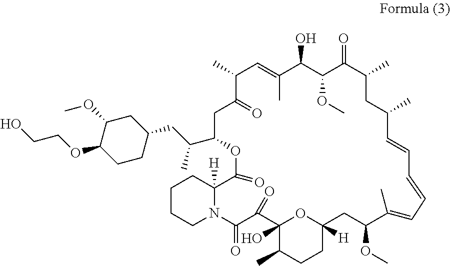 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

or a pharmaceutically acceptable salt, solvate, hydrate, cocrystal, or prodrug thereof. Everolimus is a commercially-available mTOR kinase inhibitor. Everolimus is a rapamycin analog that binds to FKBP12, and is known to partially inhibit mTOR through allosteric binding to mTORC1, with resulting clinical efficacy in cancer. The preparation and properties of everolimus are described in U.S. Pat. No. 9,079,921, the disclosures of which are incorporated by reference herein.

In an embodiment, the mTOR inhibitor is temsirolimus, also known as CCI-779 (Formula (4)):

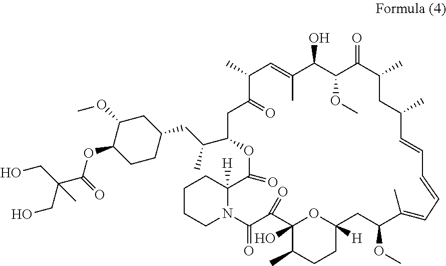 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C]

or a pharmaceutically acceptable salt, solvate, hydrate, cocrystal, or prodrug thereof. Temsirolimus is a commercially-available mTOR kinase inhibitor. The preparation, properties, and uses of temsirolimus and its derivatives and analogs are described in U.S. Pat. Nos. 5,362,718; 8,026,276; 8,299,116; 8,455,539; 8,722,700; 8,791,097; and RE44,768, the disclosures of which are incorporated by reference herein.

In an embodiment, the mTOR inhibitor is sirolimus, also known as rapamycin (Formula (5)):

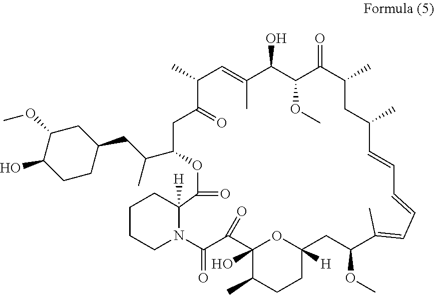 [SMILES: CO[C@H]1C[C@@H]2CC[C@@H](C)[C@@](O)(O2)C(=O)C(=O)N2CCCC[C@H]2C(=O)O[C@H](C(C)C[C@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

or a pharmaceutically acceptable salt, solvate, hydrate, cocrystal, or prodrug thereof. Sirolimus is a commercially-available mTOR kinase inhibitor. The preparation of sirolimus and its derivatives are described in U.S. Pat. Nos. 4,316,885; 5,169,851; 5,023,262; and U.S. Pat. No. 5,023,263; the disclosures of which are incorporated by reference herein.

In an embodiment, the mTOR inhibitor or PI3K inhibitor is omipalisib, which has the chemical name 2,4-difluoro-N-(2-methoxy-5-(4-(pyridazin-4-yl)quinolin-6-yl)pyridin-3-yl)benzenesulfonamide, and is also known as GSK2126458 (Formula (6)):

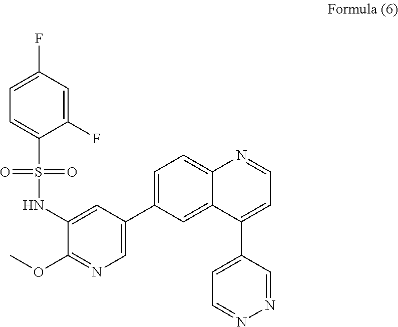 [SMILES: COc1ncc(-c2ccc3nccc(-c4ccnnc4)c3c2)cc1NS(=O)(=O)c1ccc(F)cc1F]

or a pharmaceutically acceptable salt, solvate, hydrate, cocrystal, or prodrug thereof. Omipalisib is an inhibitor of PI3K-α, -γ, and -δ as well as mTORC1/2. Knight, et al. ACS Med. Chem. Lett. 2010, 1, 39-43. Omipalisib is commercially available.

In an embodiment, the mTOR inhibitor or PI3K inhibitor is dactolisib, which has the chemical name 2-methyl-2-(4-(3-methyl-2-oxo-8-(quinolin-3-yl)-2,3-dihydroimidazo[4,5-c]quinolin-1-yl)phenyl)propanenitrile, and is also known as BEZ235 or NVP-BEZ235 (Formula (7)):

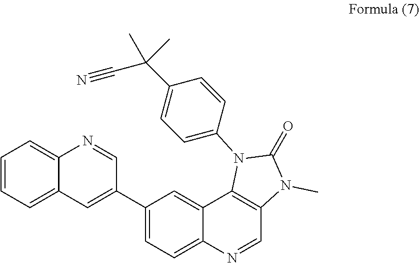 [SMILES: Cn1c(=O)n(-c2ccc(C(C)(C)C#N)cc2)c2c3cc(-c4cnc5ccccc5c4)ccc3ncc21]

or a pharmaceutically acceptable salt, solvate, hydrate, cocrystal, or prodrug thereof. Dactolisib is an inhibitor of PI3K-α, -γ, and -δ, as well as an inhibitor of mTORC1/2, and is active against preclinical models of cancer. Maira, et al., Mol. Cancer Ther. 2008, 7, 1851-63; Roper, et al. PLoS One 2011, 6, e25132. Dactolisib is available commercially.

In an embodiment, the PI3K inhibitor is pictilisib, which has the chemical name 2-(1H-indazol-4-yl)-6-((4-(methylsulfonyl)piperazin-1-yl)methyl)-4-morpholinothieno[3,2-d]pyrimidine, and is also known as GDC-0941 (Formula (8)):

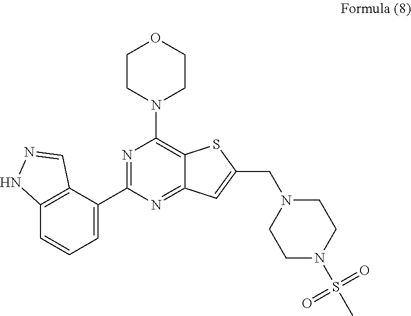 [SMILES: CS(=O)(=O)N1CCN(Cc2cc3nc(-c4cccc5[nH]ncc45)nc(N4CCOCC4)c3s2)CC1]

or a pharmaceutically acceptable salt, solvate, hydrate, cocrystal, or prodrug thereof. Pictilisib is a potent inhibitor of PI3K-α/δ that has been clinically evaluated and is commercially available. Folkes, et al., J. Med. Chem. 2008, 51, 5522-32; Sarker, et al., Clin. Cancer Res. 2015, 21, 77-86.

In an embodiment, the PI3K inhibitor is buparlisib, which has the chemical name 5-(2,6-dimorpholinopyrimidin-4-yl)-4-(trifluoromethyl)pyridin-2-amine, and which is also known as BKM120 and NVP-BKM120 (Formula (9)):

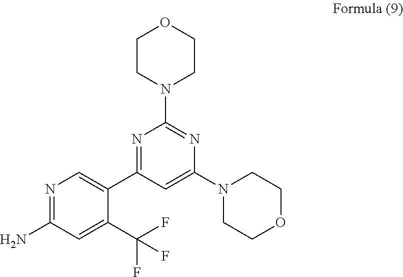 [SMILES: Nc1cc(C(F)(F)F)c(-c2cc(N3CCOCC3)nc(N3CCOCC3)n2)cn1]

or a pharmaceutically acceptable salt, solvate, hydrate, cocrystal, or prodrug thereof. Buparlisib is a potent inhibitor of PI3K-α/β/γ/δ and is commercially available. Geuna, et al., Exp. Opin. Investig. Drugs 2015, 24, 421-31; Burger, et al., ACS Med. Chem. Lett., 2011, 2, 774-779.

In an embodiment, the PI3K inhibitor is duvelisib, which has the chemical name 1(2H)-isoquinolinone, 8-chloro-2-phenyl-3-[(1S)-1-(9H-purin-6-ylamino)ethyl]-, and is also known as IPI-145 and INK1197 (Formula (10)):

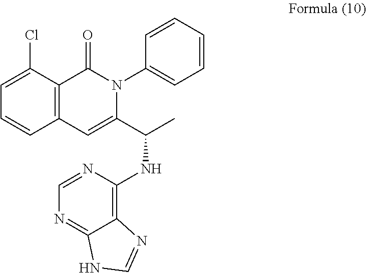 [SMILES: C[C@H](Nc1ncnc2[nH]cnc12)c1cc2cccc(Cl)c2c(=O)n1-c1ccccc1]

or a pharmaceutically acceptable salt, solvate, hydrate, cocrystal, or prodrug thereof. Duvelisib is a PI3K-γ/δ inhibitor that has been clinically evaluated and is commercially available. Balakrishnan, et al., Leukemia 2015, 29, 1811-22; Flinn, et al., Blood, 2014, 124, 802, and O'Brien, et al., Blood, 2014, 124, 3334. The preparation and properties of duvelisib, as well as other PI3K inhibitors useful in the present compositions and methods are disclosed in U.S. Pat. No. 8,193,182 and U.S. Pat. No. 8,569,323, and U.S. Patent Application Publication Nos. 2012/0184568 A1, 2013/0344061 A1, and 2013/0267521 A1, the disclosures of which are incorporated by reference herein.

In an embodiment, the PI3K inhibitor is copanlisib, which has the chemical name 5-pyrimidinecarb oxamide, 2-amino-N-[2,3-dihydro-7-methoxy-8-[3-(4-morpholinyl)propoxy]imidazo[1,2-c]quinazolin-5-yl]-, and is also known as BAY 80-6946 (Formula (11)):

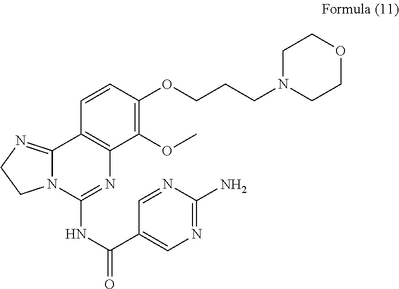 [SMILES: COc1c(OCCCN2CCOCC2)ccc2c1N=C(NC(=O)c1cnc(N)nc1)N1CCN=C21]

or a pharmaceutically acceptable salt, solvate, hydrate, cocrystal, or prodrug thereof. Copanlisib is a PI3K-α/δ inhibitor that has been clinically evaluated and is commercially available. Elster, et al., Breast Cancer Res. Treat. 2015, 149, 373-83.

In an embodiment, the PI3K inhibitor is idelalisib, which has the chemical name (S)-2-(1-((9H-purin-6-yl)amino)propyl)-5-fluoro-3-phenylquinazolin-4(3H)-one, and is also known as CAL-101 or GS-1101 (Formula (12)):

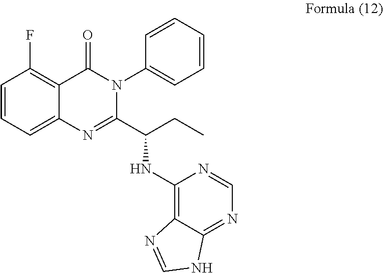 [SMILES: CC[C@H](Nc1ncnc2[nH]cnc12)c1nc2cccc(F)c2c(=O)n1-c1ccccc1]

or a pharmaceutically acceptable salt, solvate, hydrate, cocrystal, or prodrug thereof. Idelalisib is a selective PI3K-δ inhibitor and is commercially available. Lannutti, et al., Blood 2011, 117, 591-94. Idelalisib and other PI3K-δ inhibitors suitable for use in the present compositions and methods are disclosed in U.S. Pat. No. 7,932,260 and U.S. Pat. No. 8,207,153, the disclosures of which are incorporated by reference herein.

### Other Active Pharmaceutical Ingredients

In an embodiment, the invention includes a method of treating a cancer in a human subject with an inherited non-synonymous single nucleotide polymorphism at codon 47 in a TP53 gene comprising administering a therapeutically effective dose of an active pharmaceutical ingredient to the human subject, wherein the active pharmaceutical ingredient is a platinum drug. The active pharmaceutical ingredient may be any platinum drug known in the art, as described e.g. in Kelland, Nature Rev. Cancer 2007, 7, 573-84. In particular, it is one of the platinum drugs described in more detail in the following paragraphs.

In an embodiment, the active pharmaceutical ingredient is a DNA crosslinking drug, such as alkylating agents, platinum drugs, mitomycin C and furocoumarins, and psoralens. In an embodiment, the DNA crosslinking drug is a platinum drug. In an embodiment, the platinum drug is selected from the group consisting of cisplatin, carboplatin, oxaliplatin, satraplatin, picoplatin, nedaplatin, triplatin tetranitrate, lipoplatin (liposomal cisplatin), combinations thereof, and pharmaceutically acceptable salts, solvates, hydrates, cocrystals, or prodrugs thereof. The properties of cisplatin, carboplatin, oxaliplatin, satraplatin, picoplatin, nedaplatin, triplatin tetranitrate, and lipoplatin are known to those of ordinary skill in the art, and the active pharmaceutical ingredients and formulated products are commercially available. Wheate, et al., Dalton Trans. 2010, 39, 8113-27; Apps, et al., Endocrine Related Cancer 2015, 22, 219-233.

In an embodiment, the platinum drug is cisplatin, which has the chemical name (SP-4-2)-diamminedichloroplatinum(II) (Formula (13)):

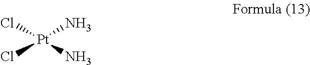

or a pharmaceutically acceptable salt, solvate, hydrate, cocrystal, or prodrug thereof. The preparation and properties of cisplatin are described in, e.g., von Hoff and Rozencweig, Adv. Pharmacol. & Chemotherapy 1979, 16, 273-294.

In an embodiment, the platinum drug is carboplatin, which has the chemical name cis-diammine(cyclobutane-1,1-dicarboxylate-O,O′)platinum(II) (Formula (14)):

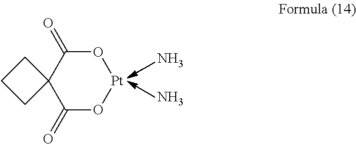

or a pharmaceutically acceptable salt, solvate, hydrate, cocrystal, or prodrug thereof. The preparation and properties of carboplatin are described in U.S. Pat. No. 4,140,707, the disclosure of which is incorporated by reference herein.

In an embodiment, the platinum drug is oxaliplatin, which has the chemical name [(1R,2R)-cyclohexane-1,2-diamine](ethanedioato-O,O′)platinum(II) or cis-[(1R,2R)-1,2-cyclohexanediamine-N,N] [oxalato(2−)-O₁O] platinum (Formula (15)):

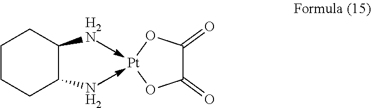

or a pharmaceutically acceptable salt, solvate, hydrate, cocrystal, or prodrug thereof. The preparation and properties of oxaliplatin are described in U.S. Pat. Nos. 4,169,846; 5,420,319; and U.S. Pat. No. 5,716,988, the disclosures of which are incorporated by reference herein.

In an embodiment, the platinum drug is satraplatin, which has the chemical name (OC-6-43)-bis(acetato)amminedichloro(cyclohexylamine)platinum or bis(acetato) ammine dichloro (cyclohexylamine) platinum(IV) (Formula (16)):

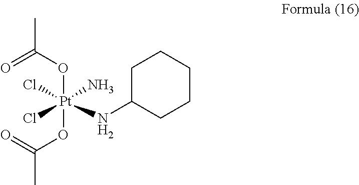

or a pharmaceutically acceptable salt, solvate, hydrate, cocrystal, or prodrug thereof. The preparation and properties of satraplatin are described in U.S. Pat. Nos. 5,072,011; 5,244,919; 6,518,428, the disclosures of which are incorporated by reference herein.

In an embodiment, the platinum drug is picoplatin, which has the chemical name azane; 2-methylpyridine; platinum(2+); dichloride (Formula (17)):

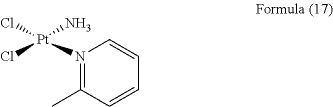

or a pharmaceutically acceptable salt, solvate, hydrate, cocrystal, or prodrug thereof. The preparation and properties of picoplatin are described in U.S. Pat. No. 5,665,771 and U.S. Pat. No. 6,518,428; the disclosures of which are incorporated by reference herein.

In an embodiment, the platinum drug is nedaplatin, which has the chemical name diammine[(hydroxy-κO)acetato(2−)-κO]platinum (Formula (18)):

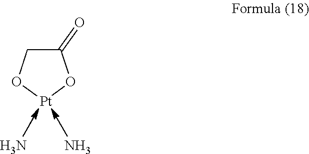

or a pharmaceutically acceptable salt, solvate, hydrate, cocrystal, or prodrug thereof. Nedaplatin has been described in Alberts, et al., Cancer Chemother. Pharmacol. 1997, 39, 493-497 and Wheate, et al., Dalton Trans. 2010, 39, 8113-8127.

In an embodiment, the platinum drug is triplatin or a pharmaceutically acceptable salt, solvate, hydrate, cocrystal, or prodrug thereof. In an embodiment, the platinum drug is triplatin tetranitrate (Formula (19)):

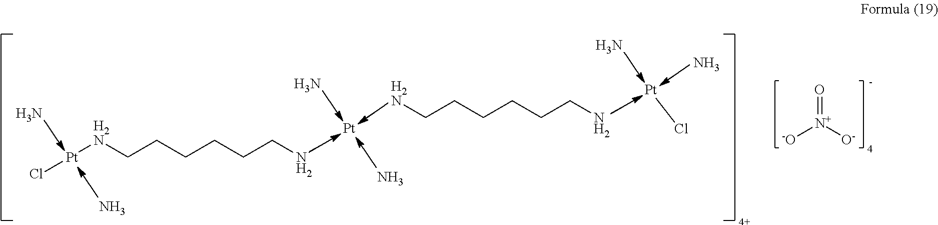

In an embodiment, the platinum drug is lipoplatin, which is a nanoparticle containing lipids and cisplatin. The clinical efficacy of lipoplatin is described in Stathopoulos, et al., Cancer Chemotherapy & Pharmacol. 2011, 68, 945-950. The preparation, properties, and uses of lipoplatin are described in U.S. Pat. No. 6,511,676, the disclosure of which is incorporated by reference herein.

### Pharmaceutical Compositions

In a preferred embodiment, an active pharmaceutical ingredient or combination of active pharmaceutical ingredients is provided as a pharmaceutically acceptable composition.

In some embodiments, the concentration of each of the chemotherapeutic active pharmaceutical ingredients provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of each of the chemotherapeutic active pharmaceutical ingredients provided in the pharmaceutical compositions of the invention is greater than 90%, 80%, 70%, 60%, 50%, 40%, 30%, 20%, 19.75%, 19.50%, 19.25%, 19%, 18.75%, 18.50%, 18.25%, 18%, 17.75%, 17.50%, 17.25%, 17%, 16.75%, 16.50%, 16.25%, 16%, 15.75%, 15.50%, 15.25%, 15%, 14.75%, 14.50%, 14.25%, 14%, 13.75%, 13.50%, 13.25%, 13%, 12.75%, 12.50%, 12.25%, 12%, 11.75%, 11.50%, 11.25%, 11%, 10.75%, 10.50%, 10.25%, 10%, 9.75%, 9.50%, 9.25%, 9%, 8.75%, 8.50%, 8.25%, 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each of the chemotherapeutic active pharmaceutical ingredients provided in the pharmaceutical compositions is in the range from about 0.0001%, to about 50%, about 0.001%, to about 40%, about 0.01%, to about 30%, about 0.02%, to about 29%, about 0.03%, to about 28%, about 0.04%, to about 27%, about 0.05%, to about 26%, about 0.06%, to about 25%, about 0.07%, to about 24%, about 0.08%, to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of each of the chemotherapeutic active pharmaceutical ingredients provided in the pharmaceutical compositions is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07%, to about 2%, about 0.08%, to about 1.5%, about 0.09%, to about 1%, about 0.1%, to about 0.9% w/w, w/v or v/v of the pharmaceutical composition.

In some embodiments, the amount of each of the chemotherapeutic active pharmaceutical ingredients provided in the pharmaceutical compositions is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of each of the chemotherapeutic active pharmaceutical ingredients provided in the pharmaceutical compositions is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the chemotherapeutic active pharmaceutical ingredients according to the invention is effective over a wide dosage range. For example, in the treatment of adult humans, dosages independently range from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

In an embodiment, the molar ratio of two active pharmaceutical ingredients in the pharmaceutical compositions is in the range from 10:1 to 1:10, preferably from 2.5:1 to 1:2.5, and more preferably about 1:1. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20.

In a preferred embodiment, the pharmaceutical compositions of the invention are for use in the treatment of cancer. In a preferred embodiment, the pharmaceutical compositions of the invention are for use in the treatment of a cancer selected from the group consisting of bladder cancer, squamous cell carcinoma including head and neck cancer, pancreatic ductal adenocarcinoma, pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, esophageal cancer, testicular cancer, gynecological cancer, thyroid cancer, acquired immune deficiency syndrome (AIDS)-related lymphoma, Kaposi's sarcoma, viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

Described below are non-limiting pharmaceutical compositions and methods for preparing the same.

### Pharmaceutical Compositions for Oral Administration

In preferred embodiments, the invention provides a pharmaceutical composition for oral administration containing the active pharmaceutical ingredient or combination of active pharmaceutical ingredients, and a pharmaceutical excipient suitable for oral administration.

In some embodiments, the invention provides a solid pharmaceutical composition for oral administration containing: (i) an effective amount of an active pharmaceutical ingredient or combination of active pharmaceutical ingredients, and (ii) a pharmaceutical excipient suitable for oral administration. In selected embodiments, the composition further contains (iii) an effective amount of a third active pharmaceutical ingredient and optionally (iv) an effective amount of a fourth active pharmaceutical ingredient.

In some embodiments, the pharmaceutical composition may be a liquid pharmaceutical composition suitable for oral consumption. Pharmaceutical compositions of the invention suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The invention further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-201aurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the invention and to minimize precipitation of the compound of the invention. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, E-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In preferred embodiments, the invention provides a pharmaceutical composition for injection containing a chemotherapeutic active pharmaceutical ingredient or combination of chemotherapeutic active pharmaceutical ingredients, and a pharmaceutical excipient suitable for injection.

The forms in which the compositions of the invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating a chemotherapeutic active pharmaceutical ingredient or combination of chemotherapeutic active pharmaceutical ingredients in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In preferred embodiments, the invention provides a pharmaceutical composition for transdermal delivery containing a chemotherapeutic active pharmaceutical ingredient or combination of chemotherapeutic active pharmaceutical ingredients, and a pharmaceutical excipient suitable for transdermal delivery.

Compositions of the invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the invention employs transdermal delivery devices (“patches”). Such transdermal patches may be used to provide continuous or discontinuous infusion of a chemotherapeutic active pharmaceutical ingredient or combination of chemotherapeutic active pharmaceutical ingredients in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and U.S. Pat. No. 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of a chemotherapeutic active pharmaceutical ingredient or combination of chemotherapeutic active pharmaceutical ingredients or a pharmaceutical composition thereof can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The chemotherapeutic active pharmaceutical ingredient or combination of chemotherapeutic active pharmaceutical ingredients can also be administered intraadiposally or intrathecally.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g. PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. The chemotherapeutic active pharmaceutical ingredient or combination of chemotherapeutic active pharmaceutical ingredients may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. The chemotherapeutic active pharmaceutical ingredient or combination of chemotherapeutic active pharmaceutical ingredients may additionally be administered intravascularly from a balloon used during angioplasty. Extravascular administration of a chemotherapeutic active pharmaceutical ingredient or combination of chemotherapeutic active pharmaceutical ingredients via the pericard or via advential application of formulations of the invention may also be performed to decrease restenosis.

Exemplary parenteral administration forms include solutions or suspensions of active compound in sterile aqueous solutions, for example, aqueous propylene glycol or dextrose solutions. Such dosage forms can be suitably buffered, if desired.

The invention also provides kits. The kits include a chemotherapeutic active pharmaceutical ingredient or combination of chemotherapeutic active pharmaceutical ingredients, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, a chemotherapeutic active pharmaceutical ingredient or combination of chemotherapeutic active pharmaceutical ingredients are provided as separate compositions in separate containers within the kit. In selected embodiments, a chemotherapeutic active pharmaceutical ingredient or combination of chemotherapeutic active pharmaceutical ingredients are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

In some embodiments, the invention provides a kit comprising a composition comprising a therapeutically effective amount of a chemotherapeutic active pharmaceutical ingredient or combination of chemotherapeutic active pharmaceutical ingredients or a pharmaceutically acceptable salt, solvate, hydrate, cocrystal, or prodrug thereof. These compositions are typically pharmaceutical compositions. The kit is for co-administration of the chemotherapeutic active pharmaceutical ingredient or combination of chemotherapeutic active pharmaceutical ingredients, either simultaneously or separately.

In some embodiments, the invention provides a kit comprising (1) a composition comprising a therapeutically effective amount of a chemotherapeutic active pharmaceutical ingredient or combination of chemotherapeutic active pharmaceutical ingredients or a pharmaceutically acceptable salt, solvate, hydrate, cocrystal, or prodrug thereof, and (2) a diagnostic test for determining whether a patient's cancer is a particular subtype of a cancer. Any of the foregoing diagnostic methods may be utilized in the kit.

The kits described above are preferably for use in the treatment of the diseases and conditions described herein. In a preferred embodiment, the kits are for use in the treatment of cancer. In preferred embodiments, the kits are for use in treating solid tumor cancers.

In a preferred embodiment, the kits of the invention are for use in the treatment of cancer. In a preferred embodiment, the kits of the invention are for use in the treatment of a cancer selected from the group consisting of bladder cancer, squamous cell carcinoma including head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thyoma, prostate cancer, colorectal cancer, ovarian cancer, acute myeloid leukemia, thymus cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, renal cancer, kidney cancer, liver cancer, ovarian cancer, esophageal cancer, testicular cancer, gynecological cancer, thyroid cancer, acquired immune deficiency syndrome (AIDS)-related cancers (e.g., lymphoma and Kaposi's sarcoma), viral-induced cancer, glioblastoma, esophageal tumors, hematological neoplasms, non-small-cell lung cancer, chronic myelocytic leukemia, diffuse large B-cell lymphoma, esophagus tumor, follicle center lymphoma, head and neck tumor, hepatitis C virus infection, hepatocellular carcinoma, Hodgkin's disease, metastatic colon cancer, multiple myeloma, non-Hodgkin's lymphoma, indolent non-Hodgkin's lymphoma, ovary tumor, pancreas tumor, renal cell carcinoma, small-cell lung cancer, stage IV melanoma, chronic lymphocytic leukemia, B-cell acute lymphoblastic leukemia (ALL), mature B-cell ALL, follicular lymphoma, mantle cell lymphoma, and Burkitt's lymphoma.

### Dosages and Dosing Regimens

The amounts of the pharmaceutical compositions administered will be dependent on the human or mammal being treated, the severity of the disorder or condition, the rate of administration, the disposition of the active pharmaceutical ingredients and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of the pharmaceutical compositions and active pharmaceutical ingredients may be provided in units of mg/kg of body mass or in mg/m² of body surface area.

In some embodiments, a pharmaceutical composition or active pharmaceutical ingredient is administered in a single dose. Such administration may be by injection, e.g., intravenous injection, in order to introduce the active pharmaceutical ingredient quickly. However, other routes, including the preferred oral route, may be used as appropriate. A single dose of a pharmaceutical composition may also be used for treatment of an acute condition.

In some embodiments, a pharmaceutical composition or active pharmaceutical ingredient is administered in multiple doses. In a preferred embodiment, a pharmaceutical composition is administered in multiple doses. Dosing may be once, twice, three times, four times, five times, six times, or more than six times per day. Dosing may be once a month, once every two weeks, once a week, or once every other day. In other embodiments, a pharmaceutical composition is administered about once per day to about 6 times per day. In some embodiments, a pharmaceutical composition is administered once daily, while in other embodiments, a pharmaceutical composition is administered twice daily, and in other embodiments a pharmaceutical composition is administered three times daily.

Administration of the active pharmaceutical ingredients of the invention may continue as long as necessary. In selected embodiments, a pharmaceutical composition is administered for more than 1, 2, 3, 4, 5, 6, 7, 14, or 28 days. In some embodiments, a pharmaceutical composition is administered for less than 28, 14, 7, 6, 5, 4, 3, 2, or 1 day. In some embodiments, a pharmaceutical composition is administered chronically on an ongoing basis—e.g., for the treatment of chronic effects. In some embodiments, the administration of a pharmaceutical composition continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of an active pharmaceutical ingredient disclosed herein is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of an active pharmaceutical ingredient disclosed herein is about 25 mg, about 50 mg, about 75 mg, about 100 mg, about 125 mg, about 150 mg, about 175 mg, about 200 mg, about 225 mg, or about 250 mg.

In some embodiments, an effective dosage of an active pharmaceutical ingredient disclosed herein is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of an active pharmaceutical ingredient disclosed herein is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, an effective dosage of an active pharmaceutical ingredient disclosed herein is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg. In some embodiments, an effective dosage of an active pharmaceutical ingredient disclosed herein is about 25 mg, about 50 mg, about 75 mg, about 100 mg, about 125 mg, about 150 mg, about 175 mg, about 200 mg, about 225 mg, or about 250 mg.

In some embodiments, an active pharmaceutical ingredient is administered at a dosage of 10 to 200 mg BID, including 50, 60, 70, 80, 90, 100, 150, or 200 mg BID. In some embodiments, an active pharmaceutical ingredient is administered at a dosage of 10 to 500 mg BID, including 1, 5, 10, 15, 25, 50, 75, 100, 150, 200, 300, 400, or 500 mg BID.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of the combination of the active pharmaceutical ingredient may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1

### Identification of Spontaneous Cancer in S47 Mice

In order to study the influence of the S47 polymorphism on p53 function within an organism, a knock-in mouse for the S47 allele was generated. The Humanized p53 Knock-in (Hupki) targeting allele was used, which replaces mouse exons 4 through 9 with the corresponding human exons (codons 32-332). There were several reasons for this choice: Hupki p53 has been shown to be fully tumor-suppressive, transcriptionally-active, and to accurately recapitulate the activity of both human and murine p53 (Frank, et al., Mol. Cell Biol. 2011, 31, 1201-1213; Luo, et al., Oncogene 2001, 20, 320-328; Reinbold, et al., Oncogene 2008, 27, 2788-2794). Also, the codon 72 variants of p53 were successfully modeled using the Hupki platform, and information was derived from those mice that subsequently held true for human p53 codon 72 variants (Frank, et al., Mol. Cell Biol. 2011, 31, 1201-1213). Finally, a knock-in mouse for p53 substituting Serine 46 with alanine (S46A) was created using the Hupki platform, and cells from these mice recapitulated the apoptotic defect evident in human cells (Feng, et al., Cell Cycle 2006, 5, 2812-2819).

WT Hupki mice were described previously, and S47 mice were generated with the Hupki targeting construct as described (Luo, et al., Oncogene 2001, 20, 320-328) following site-directed mutagenesis to create serine at amino acid 47. All studies were performed in accordance with federal and institutional guidelines according IACUC protocols. Mice were housed in plastic cages with ad libitum diet and maintained at 22° C. with a 12-hour dark/12-hour light cycle. For irradiation experiments mice were exposed to a cesium-137 gamma irradiation source (The Wistar Institute) and tissues were harvested 4 hrs later.

The WT Hupki mouse was previously generated, and was modeled both the Proline 72 (P72) and Arginine 72 variants of p53 (Frank, et al., Mol. Cell Biol. 2011, 31, 1201-1213). Because it was shown that the S47 variant appears to occur exclusively in cis with P72, S47 ES lines using the P72 Hupki platform were generated. ES lines with successfully targeted alleles were confirmed by Southern analysis. Males with germline transmission of the targeted allele were crossed to EIIA—Cre females, and Cre-mediated excision of the neomycin resistance cassette was monitored by Southern analysis. Mice were back-crossed to C57Bl/6 for over ten generations. RNA was isolated from mouse embryonic fibroblasts (MEFs) from wild type Hupki and S47-Hupki mice and used to sequence the full-length p53 cDNA; the only difference was at codon 47, which encoded proline (CCC) in WT p53 and serine in the S47 allele (TCC, data not shown). To ensure genetic homogeneity, most studies were performed on sibling littermate mice from heterozygote crosses.

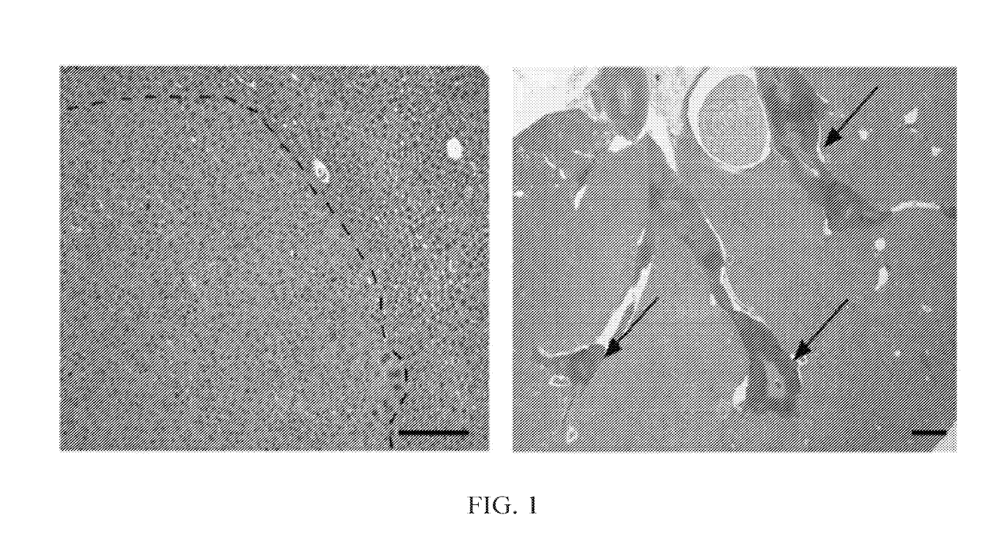

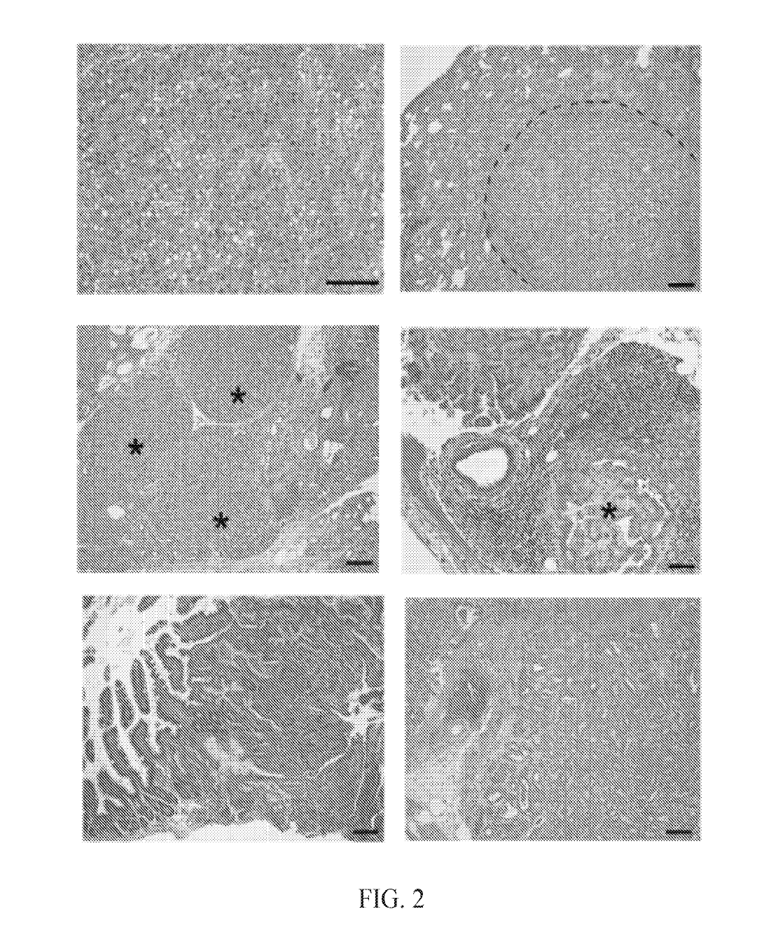

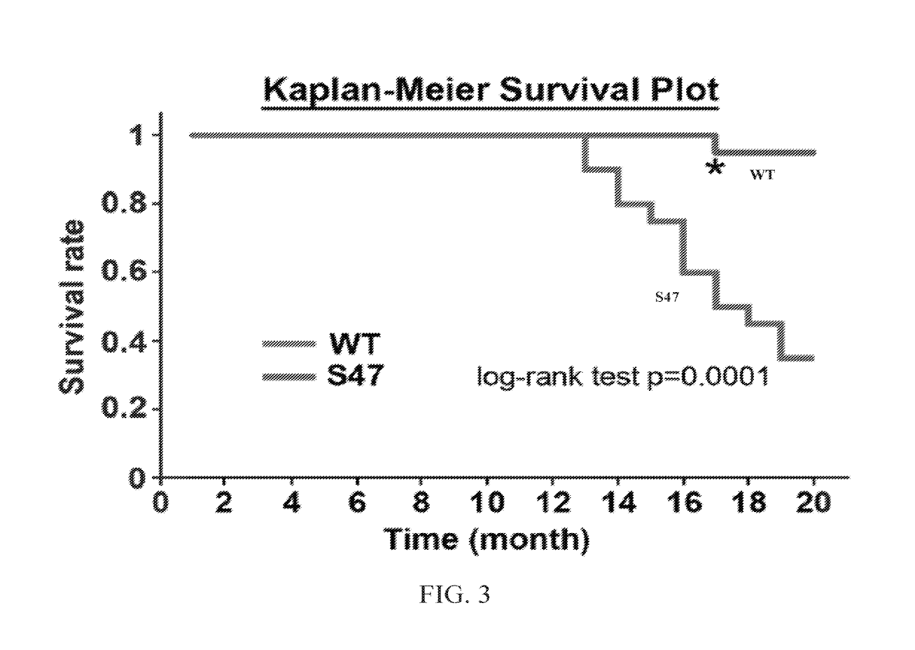

A cohort of twenty S47 and WT mice was set aside in order to analyze life expectancy. Surprisingly, a significant percentage of S47 mice developed spontaneous cancer. In all, 16/20 (80%) of the homozygous S47 mice developed cancer between twelve and eighteen months of age. These cancers were of diverse histological type, and were somewhat unusual tumor types for p53 mouse models, including histiocytic sarcoma, hepatocellular carcinoma, colorectal carcinoma, and other tumor types (FIG. 1, FIG. 2, Table 1). More surprising was the presence of metastatic lesions in a small fraction of these mice (FIG. 2, Table 1). It was also noted that the presence of prostate hyperplasia in S47 but not WT mice at eight months of age, as well as the presence of mammary nodules of undefined origin in female S47 mice. In recent analyses it was noted that three tumors that arose in S47/WT heterozygote mice (3 tumors in 12 mice; FIG. 3). Log-rank analysis of survival between WT and S47 mice revealed a statistically significant difference in survival between these WT and S47 mice (p<0.0001; FIG. 3).

TABLE 1
Cancer incidence in the S47 mouse, and in heterozygous WT/S47
mice. Data are representative of a total of twenty mice, sixteen
of which developed cancer. LL: B cell lymphoproliferative lesion.
HCC: hepatocellular carcinoma. CC: colorectal carcinoma. PA:
pancreatic adenocarcinoma. HS: histiocytic sarcoma.

| | | Age | | |
|---|---|---|---|---|
| Genotype | Tumor Type/Lesion | (Months) | Gender | Metastasis |
| S47/S47 | LL | 16 | F | − |
| S47/S47 | LL | 16 | F | − |
| S47/S47 | LL | 16 | F | − |
| S47/S47 | LL | 13 | F | − |
| S47/S47 | HCC, LL | 13 | M | + |
| S47/S47 | HCC, CC, PA | 14 | M | + |
| S47/S47 | LL | 19 | F | − |
| S47/S47 | HCC | 14 | M | − |
| S47/S47 | LL | 17 | F | − |
| S47/S47 | HCC | 15 | M | − |
| S47/S47 | Renal adenoma | 18 | M | − |
| S47/S47 | HCC | 17 | M | − |
| S47/S47 | HCC | 19 | M | − |
| WT/S47 | Ovarian | 7 | F | − |
| WT/S47 | HCC | 15 | M | − |
| WT/S47 | LL | 10 | F | − |

### Example 2

### Association of the P47S Mutation with Increased Risk of Cancer in Humans

Breast cancer data were derived from three studies of breast cancer in African-American women in the AMBER Consortium, including the Black Women's Health Study (BWHS), the Carolina Breast Cancer Study (CBSC), and Women's Circle of Health Study (WCHS) (Palmer, et al., Cancer Causes Control 2014, 25, 309-319). All studies were approved by the affiliated institutional review boards. BWHS is a prospective cohort study with participants across the U.S. enrolled by mailed questionnaires and followed with biennial and 5-year interval follow-up questionnaires. CBCS and WCHS are both case-control studies, with CBCS 1 and 2 conducted with population-based sampling and in-person interviews from 1993-2001 in 24 counties in North Carolina. WCHS, initiated in 2002 in metropolitan New York City and several counties in eastern N.J., is still ongoing in N.J. For these analyses, women were included with invasive cancer or ductal carcinoma in situ with adequate DNA available (n=3130), confirmed by pathology reports or registry records from which we also obtained data on ER status, and 3,698 controls. Genotyping was performed at the Center for Inherited Diseases (CIDR) as part of a larger project, using the Illumina HumanExome Beadchip Plus v1.1 plus 200,000 custom beadtypes. Imputation based on 1,000 Genomes data was carried out at the University of Washington. The observed:expected variance ratio, a measure of squared correlation between the imputed genotypes and the true genotypes for the imputed SNP was 0.91. Associations were examined between the imputed SNP rs1800371 in TP53 and breast cancer overall, estrogen receptor positive (ER+) cancer, and ER negative (ER−) disease. Ancestry is a recognized potential confounder of genetic associations. Study (BWHS, WCHS, or CBCS), geographic location (N.J., Northeast minus N.J., South, Midwest or West), and principal components (PCs) of the genotypes for ancestry were included in the model to account for participant ancestry. DNA source (blood, saliva, mouthwash) differed slightly by case status and thus was included as a covariate to avoid confounding. Age at case diagnosis for cases and matched controls (in 10 year groupings) was a design variable and thus included as a covariate. The odds ratios (ORs) and 95% CIs were derived from multivariable logistic regression models which adjusted for study (BWHS, WCHS, or CBCS), age (in 10 year groupings), DNA source (blood, saliva, mouthwash) and geographic location (N.J., Northeast minus N.J., South, Midwest or West) and principal components (PCs) of the genotypes for ancestry (all PCs with p<0.1 after including the covariates listed above in the model). We repeated the analyses limited to the >98% of premenopausal samples for which the maximum posterior genotype probability was >90%. A subset of samples was also genotyped in blinded manner using Taqman analysis and primers from Applied Biosystems for the rs1800371 SNP in the Wistar Institute Genomics Facility; all Taqman genotyping was 100% concordant with imputed data. All analyses were conducted using SAS 9.4 (SAS Institute, Cary, N.C.).

As previously described, data was obtained from the AMBER Consortium, which pools data from several large studies of breast cancer in African-American women (Palmer, et al., Cancer Causes Control 2014, 25, 309-319). Imputed data on the P47S polymorphism (r51800371) were available for 3,130 cases and 3,698 controls. As shown in Table 2, rs1800371 is fairly rare in this population of African-Americans, with a minor allele frequency of 1.4% in controls. There were no significant associations between rs1800371 and breast cancer risk overall, or with ER status.

TABLE 2
Associations between TP53 rs1800371 and breast cancer risk in the AMBER Consortium.

| | | All | | ER+ | | ER− | |
|---|---|---|---|---|---|---|---|
| | Controls | Cases | | Cases | | Cases | |
| SNP | n (%) | n (%) | OR (95% CI)* | n (%) | OR (95% CI)* | n (%) | OR (95% CI)* |
| | All Women | | | | | | |
| TP53 rs1800371 | | | | | | | |
| GG | 4564 (97) | 3557 (97) | 1.00 | 1930 (97) | 1.00 | 1066 (97) | 1.00 |
| GA | 122 (3) | 103 (3) | 1.09 (0.81-1.46) | 50 (2) | 0.97 (0.67-1.40) | 32 (3) | 1.13 (0.73-1.74) |
| AA | 1 (<1) | 3 (<1) | 2.48 (0.20-31.57) | 3 (1) | 4.60 (0.35-60.39) | 0 | NA |
| GA + AA | 123 | 106 | 1.10 (0.82-1.47) | 53 | 1.00 (0.70-1.43) | 32 | 1.12 (0.72-1.71) |
| Per allelle OR | | | 1.11 (0.84-1.48) | | 1.04 (0.73-1.46) | | 1.11 (0.72-1.72) |
| | | | P trend = 0.47 | | P trend = 0.84 | | P trend = 0.62 |
| | Premenopausal Women | | | | | | |
| TP53 rs1800371 | | | | | | | |
| GG | 1426 (98) | 1156 (96) | 1.00 | 599 (97) | 1.00 | 378 (97) | 1.00 |
| GA | 35 (2) | 43 (4) | 1.84 (1.11-3.07) | 20 (3) | 1.58 (0.84-2.97) | 13 (3) | 2.00 (0.95-4.20) |
| AA | 1 (<1) | 0 | NA) | 0 | NA | 0 | NA |
| GA + AA | 36 | 43 | 1.80 (1.09-3.00) | 20 | 1.55 (0.81-2.88) | 13 | 1.96 (0.91-4.01) |
| Per allelle OR | | | 1.73 (1.06-2.84) | | 1.49 (0.81-2.76) | | 1.87 (0.91-3.82) |
| | | | P trend = 0.03 | | P trend = 0.20 | | P trend = 0.09 |

*Adjusted for study (BWHS, WCHS, or CBCS), age, DNA source, geographic location and principal components (PCs) of the genotypes for ancestry.

The estrogen pathway intersects with the p53 signaling pathway, and SNPs in the p53 pathway are associated with breast cancer risk in pre-menopausal women (Bond, et al., Cancer Res. 2006, 66, 5104-5110). Because of this, the analysis was performed on the impact of S47 on pre-menopausal women as shown in Table 2. Among pre-menopausal women, 1,218 cases and 1,490 controls, the per-allele OR was 1.79 (95% CI, 1.07-2.99; p=0.03). Because the SNP was imputed from genotype data, we repeated this analysis limited to the greater than 98% of premenopausal samples for which the maximum posterior genotype probability was greater than 90%. The comparable OR was 2.07 (95% CI, 1.18-3.65; p =0.01), supporting the premise that the association is not being driven by a few poorly imputed samples. To add confidence to this analysis a subset of twenty of these samples were genotyped, and the genotyping results were 100% in concordance with imputation.

The prostate cancer cases used in this study were from two sources, the following published study (Xu, et al., Cancer Epidemiol. Biomarkers Prev. 2009, 18, 2145-2149), or the University of Pennsylvania Health System (UPHS) via the Study for Clinical Outcomes Risk and Ethnicity (SCORE). For SCORE samples, all patients seen in the UPHS clinic who were newly diagnosed within the previous 12 months with a histologically confirmed primary prostate cancer at any stage were invited to participate in SCORE. Case status was confirmed by medical records review using a standardized abstraction form. Men were excluded from this study if they reported having exposure to finasteride (Proscar) at any time prior to their prostate cancer diagnosis, were diagnosed more than twelve months prior to the date of study ascertainment, or had ever been diagnosed with cancer at any site (except non-prostate cancer skin cancer) other than their recently diagnosed prostate cancer. The Institutional Review Board of the Perelman School of Medicine at the University of Pennsylvania approved the study protocol. The African American study population from Johns Hopkins consisted of 730 prostate cancer patients undergoing treatment in the Department of Urology at Johns Hopkins Hospital. The average age at diagnosis was 57 years (median, 57 years), and the range was 34-74 years. The Institutional Review Board of Johns Hopkins University approved the study protocol. For genotyping, DNA was extracted from whole blood or non-cancerous tissue using standard methods. Samples were genotyped using Taqman assays (r51800371; AppliedBiosystems, Foster City, Calif., USA). A randomly selected subset of samples was subjected to duplicate genotyping and analysis by Sanger sequencing in a blinded fashion, with 100% concordance among duplicate samples.

Preliminary analysis of the S47 allele in African American prostate cancer in men cases suggests a trend toward increased S47 representation in prostate cancer from younger men (<54 years old), as shown in Table 3. This trend approaches but does not reach statistical significance.

TABLE 3
Association between the S47 polymorphism and the incidence
of prostate cancer in younger (<54 years old) African
American men (n = 995). OR = 1.85 (95% confidence
interval: 0.856 to 4.025); p = 0.097.

| Age | AA/AG (S47) | GG (WT) |
|---|---|---|
| >54 | 15 | 597 (2.51%) |
| <54 | 17 | 366 (4.64%) |

The P47S polymorphism can markedly impair the apoptotic function of p53, and as described above, the S47 variant is associated with an increase of almost 80% in breast cancer risk in pre-menopausal African American women. These data support the hypothesis that the S47 variant is a risk factor in cancer risk and progression in African and Hispanic-American populations.

### Example 3

### Screening of Active Pharmaceutical Ingredients

Screening studies were performed to establish the efficacy of different active pharmaceutical ingredients in tumors that exhibit the S47 variant.

The screening methods were performed as follows. S47 and wild type cells were plated at a density of 750 cells/well in a total volume of 50 μL in 384 well Greiner microplates. The cells were allowed to attach overnight at 37° C. and then dosed with 50 nL of compound serially diluted in DMSO. Following compound addition, the assay plates were incubated at 37° C. for 72 hours, followed by the addition of 5 μL of 500 mM Resazurin. The plates were incubated at 37° C. for 8-10 hours and then fluorescence was measured using a Perkin Elmer Envision plate reader (excitation at 530-560 nm, emission at 590 nm). Raw fluorescent counts were uploaded into Spotfire and normalized to doxorubicin (positive control) and DMSO (negative control) treated wells (n=12/plate). EC50 values were calculated by applying a logistic regression curve fit and expressed as μM compound that was effective in killing 50% of the cell population at 72 hours. The results are given in Table 4.

TABLE 4
Altered Cytotoxicity of Active Pharmaceutical
Ingredients Based upon S47 SNP.

| | S47 | WT | | |
|---|---|---|---|---|
| | EC50 | EC50 | | |
| Compound | (μM) | (μM) | Target Class | |
| | | | | Fold- |
| | | | | sensitive, S47 |
| OSI027 | 0.173 | 0.745 | mTOR | 4.3 |
| INK128 | 0.0095 | 0.0145 | mTORC1/C2 | 1.5 |
| GSK2126458 | 0.00046 | 0.0015 | PI3K, mTOR | 3.26 |
| GDC0941 | 0.012 | 0.028 | PI3K | 2.33 |
| | | | | Fold- |
| | | | | resistant, S47 |
| AZD6738 | >10 | 0.377 | ATR/ATM | 26 |
| Cisplatin | 0.79 | 0.348 | DNA damage | 2.3 |
| Doxorubicin | 0.292 | 0.146 | DNA damage | 2 |
| LBH589 | 0.0066 | 0.0032 | inhibitor | 2 |
| AMN107 | >10 | 0.353 | Bcr-Abl | 28 |

### Example 4

### Diagnostic Tests for P47S Polymorphism

A suitable diagnostic PCR test for rs1800371 uses TaqMan SNP Genotyping Assays, and Life Technologies catalog #4351379 for context sequence:

GTGAACCATTGTTCAATATCGTCCG[A/G]GGACAGCATCAAATCATCCATTGCT (SEQ ID NO:1), wherein [A/G] represents the variant. SEQ ID NO: 1 may be used in determining the absence or presence of rs1800371.

### Example 5

### Microarray Analysis of WT and S47 Human B cells

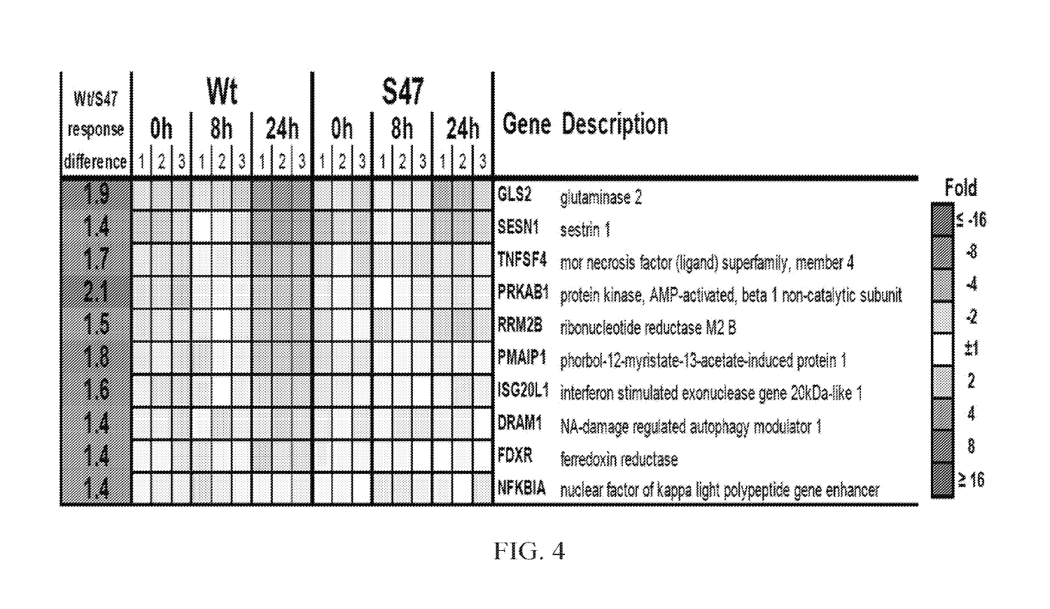

Microarray analysis was performed on WT and S47 human B cells in the presence and absence of cisplatin. This identified a subset of cisplatin-induced genes that show differences between WT and S47 cells. All of these are known p53-target genes. S47 is impaired in the transactivation of a subset of about 20 p53 target genes, at least 10 of which are involved in the control of metabolism. In particular, S47 is impaired for the ability to induce the expression of SCO2, AMPK-beta, and GLS2, and all of these influence the ability of a cell to conduct oxidative phosphorylation, the mitochondrial process for generating ATP (FIG. 4). These data suggested that the means of creating energy in WT and S47 cells may differ.

### Example 6

### Oxidative Phosphorylation in S47 Cells

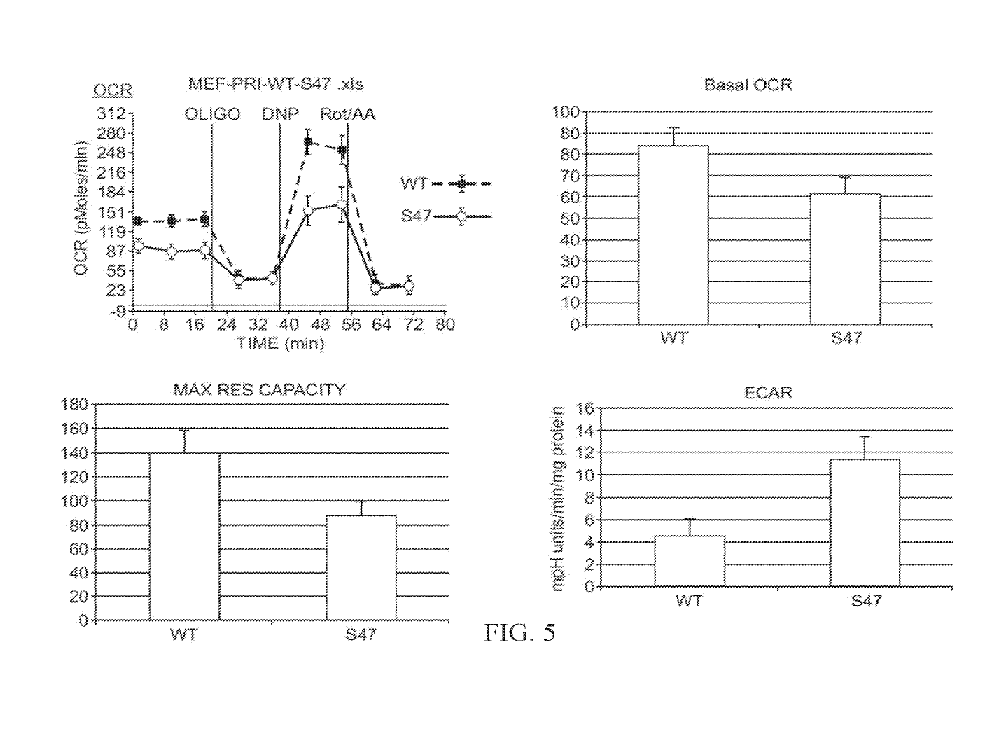

We used a Seahorse Biosciences analyzer to analyze the ability of S47 cells to conduct oxidative phosphorylation. Oxidative phosphorylation is markedly impaired in S47 cells relative to cells with WT p53 (FIG. 5; these data are normalized to cell number and protein content). This indicates that S47 cells are primed for aerobic glycolysis, instead of oxidative phosphorylation. Using aerobic glycolysis instead of oxidative phosphorylation is a key characteristic of tumor cells (the so-called “Warburg effect”). These results indicate that (1) S47 cells are primed for cancer metabolism, and (2) drugs targeting metabolism may show enhanced cytotoxicity in tumor cells that are S47 in comparison to WT cells.

### Example 7

### Microarray Analysis of WT and S47 Human B cells

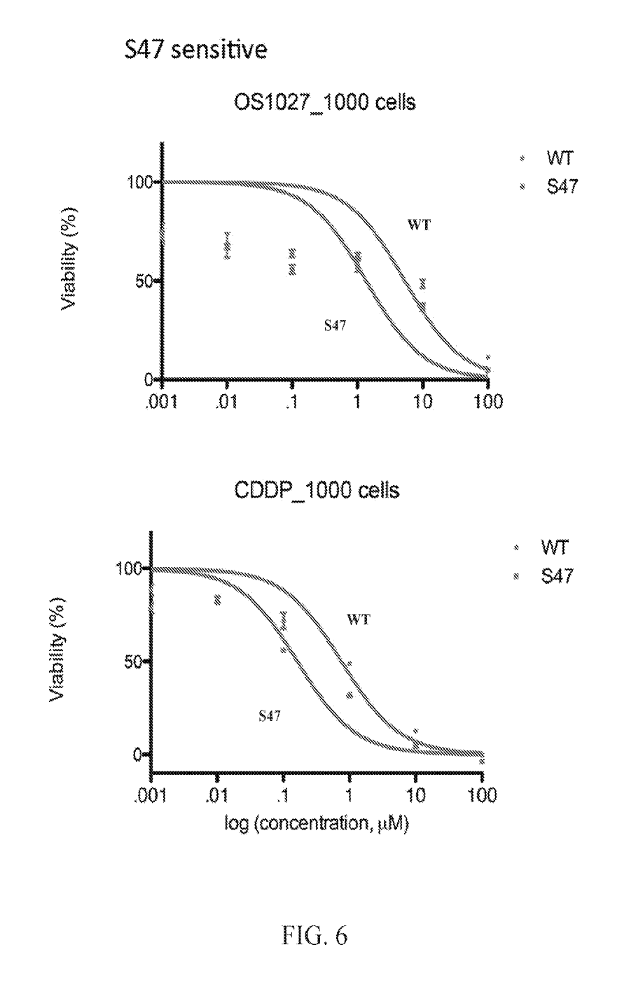

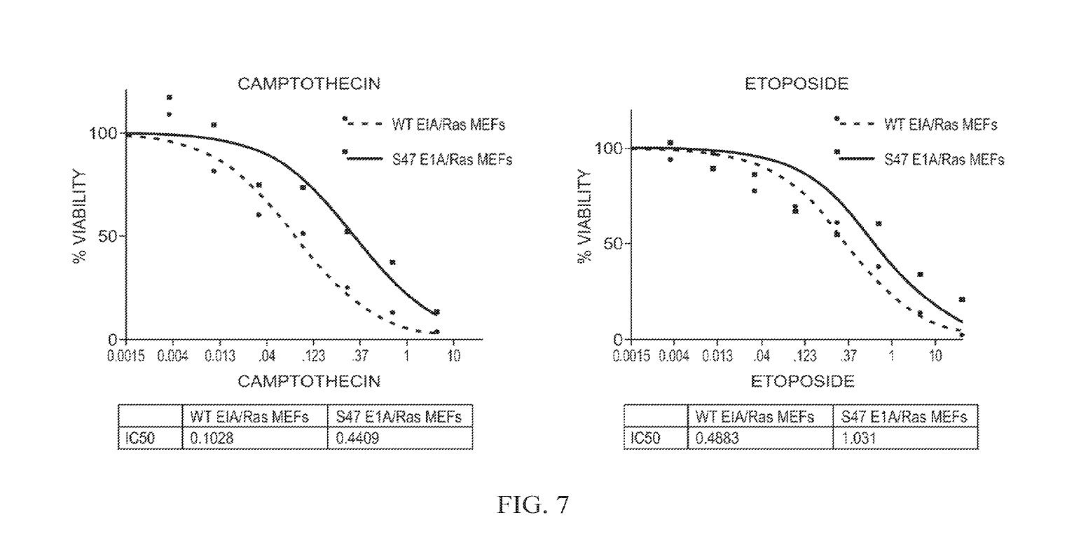

A tumorigenic (transformed) version of WT and S47 cells may be created as follows. WT and S47 cells were transfected with two oncogenes (E1A and Ras). These cell lines may be used to determine the sensitivity of S47 tumor cells to inhibitors of mTORC1/2, the major regulator of metabolism in the cell (FIG. 6). It was also found that S47 tumor cells are more sensitive to cisplatin, a DNA cross-linking agent, compared to WT cells. While S47 tumor cells are more sensitive to mTOR inhibitors and cisplatin, they are also less sensitive to other genotoxic agents, such as camptothecin and etoposide (topoisomerase inhibitors) (FIG. 7).

A number of patent and non-patent publications are cited herein in order to describe the state of the art to which this invention pertains. The entire disclosure of each of these publications is incorporated by reference herein.

While certain embodiments of the invention have been described and/or exemplified above, various other embodiments will be apparent to those skilled in the art from the foregoing disclosure. The invention is, therefore, not limited to the particular embodiments described and/or exemplified, but is capable of considerable variation and modification without departure from the scope and spirit of the appended claims.

1. A method of treating a cancer in a human subject with an inherited non-synonymous single nucleotide polymorphism (SNP) at codon 47 in a TP53 gene comprising administering a therapeutically effective dose of an active pharmaceutical ingredient to the human subject, where the subject is either homozygous or heterozygous for the SNP.

2. The method of claim 1, wherein the active pharmaceutical ingredient is selected from the group consisting of PI3K inhibitors, mTOR inhibitors, platinum drugs, and combinations thereof.

3. The method of claim 1, wherein the cancer is a cancer that does not mutate the TP53 gene.

4. The method of claim 1, wherein the cancer is selected from the group consisting of cancers that rarely mutate p53, wherein the group of cancers that rarely mutate p53 consists of melanoma, medulloblastoma, Wilms tumor, neuroblastoma, colorectal cancer, breast cancer, prostate cancer, and liver cancer.

5. The method of claim 1, wherein the human subject is of Hispanic or African-American origin.

6. The method of claim 1, wherein the active pharmaceutical ingredient is a mTOR inhibitor, and wherein the mTOR inhibitor is selected from the group consisting of trans-4-[4-amino-5-(7-methoxy-1H-indol-2-yl)imidazo[5,1-f][1,2,4]triazin-7-yl]cyclohexanecarboxylic acid (OSI-027), sapanisertib, omipalisib, dactolisib, everolimus, temsirolimus, sirolimus, and pharmaceutically acceptable salts, solvates, hydrates, cocrystals, or prodrugs thereof.

7. The method of claim 1, wherein the active pharmaceutical ingredient is a PI3K inhibitor, and wherein the PI3K inhibitor is selected from the group consisting of omipalisib, dactolisib, pictilisib, buparlisib, duvelisib, copanlisib, idelalisib, and pharmaceutically acceptable salts, solvates, hydrates, cocrystals, or prodrugs thereof.

8. The method of claim 1, wherein the active pharmaceutical ingredient is a platinum drug, and wherein the platinum drug is selected from the group consisting of cisplatin, carboplatin, oxaliplatin, satraplatin, picoplatin, nedaplatin, triplatin tetranitrate, lipoplatin (liposomal cisplatin), and pharmaceutically acceptable salts, solvates, hydrates, cocrystals, or prodrugs thereof.

9. A method performed on a biological sample from a human subject, comprising detecting a non-synonymous single nucleotide polymorphism at codon 47 in a TP53 gene in the biological sample.

10. The method according to claim 9, wherein the detecting further comprises detecting the presence of a nucleic acid sequence.

11. The method according to claim 10, wherein the detecting further comprises amplifying the nucleic acid sequence.

12. The method according to claim 11, wherein the detecting comprises quantitatively analyzing the non-synonymous single nucleotide polymorphism.

13. The method according to claim 12, wherein the quantitatively analyzing comprises quantitatively analyzing by a quantitative reverse-transcription polymerase chain reaction.

14. The method according to claim 9, wherein the biological sample is a blood sample.

15. A composition comprising therapeutically effective amounts of an active pharmaceutical ingredient selected from the group consisting of a PI3K inhibitor, a mTOR inhibitor, a platinum drug, and a combination thereof, for use in the treatment of a cancer in a human with an inherited non-synonymous single nucleotide polymorphism at codon 47 in a TP53 gene.

16. The composition of claim 15, wherein the cancer is a cancer that does not mutate the TP53 gene.

17. The composition of claim 15, wherein the cancer is selected from the group consisting of cancers that rarely mutate p53, wherein the group of cancers that rarely mutate p53 consists of melanoma, medulloblastoma, Wilms tumor, neuroblastoma, colorectal cancer, breast cancer, prostate cancer, and liver cancer.

18. (canceled)

19. The composition of claim 15, wherein the active pharmaceutical ingredient is a mTOR inhibitor, and wherein the mTOR inhibitor is selected from the group consisting of trans-4-[4-amino-5-(7-methoxy-1H-indol-2-yl)imidazo[5,1-f][1,2,4]triazin-7-yl]cyclohexanecarboxylic acid (OSI-027), sapanisertib, omipalisib, dactolisib, everolimus, temsirolimus, sirolimus, and pharmaceutically acceptable salts, solvates, hydrates, cocrystals, or prodrugs thereof.

20. The composition of claim 15, wherein the active pharmaceutical ingredient is a PI3K inhibitor, and wherein the PI3K inhibitor is selected from the group consisting of omipalisib, dactolisib, pictilisib, buparlisib, duvelisib, copanlisib, idelalisib, and pharmaceutically acceptable salts, solvates, hydrates, cocrystals, or prodrugs thereof.

21. The composition of claim 15, wherein the active pharmaceutical ingredient is a platinum drug, and wherein the platinum drug is selected from the group consisting of cisplatin, carboplatin, oxaliplatin, satraplatin, picoplatin, nedaplatin, triplatin tetranitrate, lipoplatin (liposomal cisplatin), and pharmaceutically acceptable salts, solvates, hydrates, cocrystals, or prodrugs thereof.


SEQUENCE LISTING (1 sequence)

SEQ ID NO: 1 - DNA, 51 bp, Artificial Sequence
  synthetic sequence
  misc_feature (26)..(26): where n is a or g
gtgaaccattgttcaatatcgtccgnggacagcatcaaatcatccattgct